(19) United States
(12) Patent Application Publication
(10) Pub. No.: US 2015/0258302 A1
(43) Pub. Date: Sep. 17, 2015

(54) WEARABLE DEVICE FOR COMFORTING AND COMMUNICATING WITH AUTISTIC PERSONS AND WORKING METHOD THEREOF

(71) Applicants: Siddharth Avi Chandra, Shanghai (CN); Tiger Chen Lu, Shanghai (CN); Russell Francis Hodgson Pecka, Toronto (CA); Maximilian James Wagner, Beverly Hills, MI (US); Anna Victoria Serbent, Hongqiao (CN)

(72) Inventors: Siddharth Avi Chandra, Shanghai (CN); Tiger Chen Lu, Shanghai (CN); Russell Francis Hodgson Pecka, Toronto (CA); Maximilian James Wagner, Beverly Hills, MI (US); Anna Victoria Serbent, Hongqiao (CN)

(21) Appl. No.: 14/656,108

(22) Filed: Mar. 12, 2015

(30) Foreign Application Priority Data
Mar. 14, 2014 (CN) 201410096175.7

(51) Int. Cl. A61M 21/02

(52) U.S. Cl. CPC A61M 21/02; A61M 2021/0022

(57) ABSTRACT

A personal and wearable device includes a strap, which is worn by the user, and a remote controller, which is held by a guardian or a teacher. The strap includes a built-in heart rate monitor, a built-in accelerometer and a built-in vibration motor, wherein the heart rate monitor and accelerometer are used to determine the current state of the child in real time. The accelerometer is used to monitor any sudden or repeated actions of the arms or limbs of the user in order to determine whether the user is undergoing an outburst based off the magnitude of the measured acceleration in the arm; and the vibration motor may generate vibrations, which are adjustable in strength via the remote controller. The device is compact in size and convenient to carry, and thus provides a realistic solution for the integration of autistic persons into mainstream schools, etc.

### CROSS REFERENCE TO RELATED APPLICATION

This application claims priority of Chinese patent application serial number 201410096175.7, filed Mar. 14, 2014, which is incorporated herein in its entirety by reference.

### FIELD OF THE INVENTION

The following describes a system that includes wearable device and at least one of a corresponding remote or a mobile application. The system, in one non-limiting instance, aids in calming and comforting autistic persons using localized vibration therapy. It also acts as a two-way communication system, in which the autistic person can express certain needs and simple messages. Communication between the wearable device and the remote is carried out using wireless and/or wired technology.

### BACKGROUND OF THE INVENTION

Autism is defined as a spectrum of pervasive developmental disorders with a variety of symptoms including repetitive stereotypic tendencies and communication difficulties. According to a study by the U.S. Center for Disease Control and Prevention, one out of every 68 American children has been diagnosed with Autism and tens of millions of autistic people have been diagnosed worldwide. The effected children often find it very difficult to adapt to mainstream society, often restricting their opportunities to succeed. One method of easing assimilation into mainstream society is for these children to attend mainstream schools where they can interact with other non-autistic children. However, in this environment the inabilities caused by autism are often too restricting for the autistic child. For this problem there is no effective means of solving this issue so far.

Experts generally believe that it is advantageous for autistic children to receive an education at mainstream schools rather than special schools, since increasing their exposure to social interaction helps these children learn to control and modify their behaviors. However, according to Autism experts, such as Temple Grandin, the integration of autistic children into mainstream schools can only be achieved if steps are taken to reduce environmental and social pressures. These children can only feel accepted if they have the resources to communicate and behave in a way that does not alienate them from their peers.

There are a variety of products that have been developed to mitigate the effects of Autism, many of which adopt distraction based vibration therapy. This approach is confirmed to be extremely effective in dealing with the autistic children suffering from stereotypy by distracting users from their environment with vibration stimulations, as is evident by their wide use in therapeutic environment.

In addition, to help the users communicate, and thus reduce the social gap between the autistic and non-autistic population, some devices have a customizable communication option feature. It is confirmed that the distraction based vibration therapy and customizable communication features in this device are helpful for the autistic children. However, in general, current products are expensive, large in size, and only capable of preforming one of these two functions., This makes them unrealistic tools in mainstreaming autistic children.

### SUMMARY OF THE INVENTION

The following describes a wearable device for an autistic person, which not only comforts a user suffering from anxiety with vibration therapy, but also eases the communication difficulties between the user and his or her guardian or teacher. It utilizes wireless (or wired) technology, it is light in weight, convenient to carry and helpful for the users attempting to integrate into mainstream schools.

The system described herein, in one instance, solves one or more of the issues faced by autistic children. The system provides a personal wearable device for an autistic person. The device includes a strap configured for one or more of a wrist, an ankle, a forearm, a shoulder, a neck, and/or elsewhere on the body. The strap, in one instance, secures the device to the user. The system further includes a remote controller, which can be held by a guardian or a teacher. The strap includes a built-in heart rate monitor, accelerometer and vibration motor. The heart rate monitor is used to analyze the heart rate of the user in real time and send the heart rate to the remote controller via a wireless (or wired) signal transceiver. The heart rate can be displayed by an indicator on the remote controller. The system further includes an accelerometer. The accelerometer can be used to monitor the sudden and repeated actions of the user, e.g., in order to determine whether the user is undergoing an episode of anxiety or engaging in stereotypy. The data from all of these inputs can also be processed by an onboard microcomputer, e.g., to automatically determine whether the child is experiencing an episode of anxiety or stereotypy. From this information, the vibrations can be automatically triggered if no response is detected from the remote or mobile application. Settings, at the discretion of the guardian via the remote control or mobile application, may also be adjusted according to the magnitude of the acceleration and the strength of the vibrations as desired by the guardian. Suitable wireless communication could be Bluetooth, radio-frequency, or any other form of inter device communication. This wireless communication could take place between either the strap and a mobile application, or strap and the remote.

The following summarizes a non-limiting embodiment of the wearable device.

An upper strap and a lower strap are connected by a latch device to form a detachable complete strap. The strap could be made from a high polymer material. The upper strap is provided with built-in Bluetooth or a radio-frequency antenna and may send information to the remote controller and simultaneously receive instructions sent by the remote controller. The lower strap is provided with the heart rate monitor on an inner side surface in contact with the skin of the user, and the heart rate monitor may analyze the heart rate of the user in real time and send the heart rate to a heart rate indicator of the remote controller through the radio-frequency antenna to be displayed.

The upper strap is provided with the built-in accelerometer, which may monitor the sudden and repeated actions (such as sudden onset of anxious behavior and repeated stereotyped behaviors) of the user, thus detect the occurrence of stereotypy. The accelerometer, working in conjunction with the heart rate monitor measures the increase in heart rate and occurrence of any unusual behavior. The baseline values of the heart rate and acceleration will be individually calculated and constantly adjusted according to physical state of the child in order to reduce false alarms as much as possible. The upper strap is provided with the built-in miniature vibration motor, which may generate vibrations under the trigger of the remote controller or automatically. The upper surface of the upper strap is provided with buttons, which may be pressed to trigger corresponding lights on the remote controller or mobile application. A panel of the remote controller is provided with buttons, lights or displays of the same color as those on the strap. The panel of the remote controller also includes a vibration adjusting button, which may be used to adjust the intensity of the built-in vibration motor of the strap.

For the wearable device outlined by the present invention, the strap is worn by the user, and the remote controller is held by the guardian or teacher. To use the communication feature, the user presses the button on the strap, which causes the corresponding LED on the remote controller to light. This demonstrates a need to the guardian; and when the user experiences an irregularity of recorded physiological signals, the strap informs the guardian who controls the strap remotely and has the option to generate vibrations in order to comfort the user.

The working method of the device comprises of the strap, which can be worn at a wrist but is not limited to such use; the heart rate monitor, which is used to analyze the heart rate of the user in real time; the accelerometer monitors the actions of the arm of the user; and the data from these sensors is used to determine whether the user has is experiencing an episode of anxiety or frustration according to the movement measured by the accelerometer and the heart rate measured by the heart rate monitor. If the child is in this state then an alarm signal is sent to the remote controller, and the guardian or teacher may choose to snooze the alarm as a false positive or, apply the vibrations, in the case that no response is received from the guardian after a set amount of time the strap will automatically deliver the vibrations

The present invention is beneficial for the following reasons: it integrates the interference vibration therapy and communication function, in addition to being compact in size and convenient to carry. The automatic detection and reaction function can also calm the child down when the guardian or teacher is not present. It provides a solution for the autistic population to fit into the mainstream schools and may considerably improve their ways of life.

### BRIEF DESCRIPTION OF THE DRAWINGS

FIG. 1 is a schematic view of an example of the external structure of a wearable device.

FIG. 2 is a schematic view of an example of the external structure of a remote controller.

FIG. 3 is a schematic perspective view of the example in FIG. 1 with the housing not visible.

FIG. 4 is another schematic perspective view of the example in FIG. 1 with the housing not visible.

FIG. 5 is yet another schematic perspective view of the example in FIG. 1 with the housing not visible.

FIG. 6 is an exploded view of the example in FIG. 1.

### DETAILED DESCRIPTION OF THE EMBODIMENTS

Examples embodiments of the individual parts of the invention are illustrated below. The embodiments can be implemented based on a technical solution, providing an example implementation and an operation process, however, the system is not limited to the following embodiments.

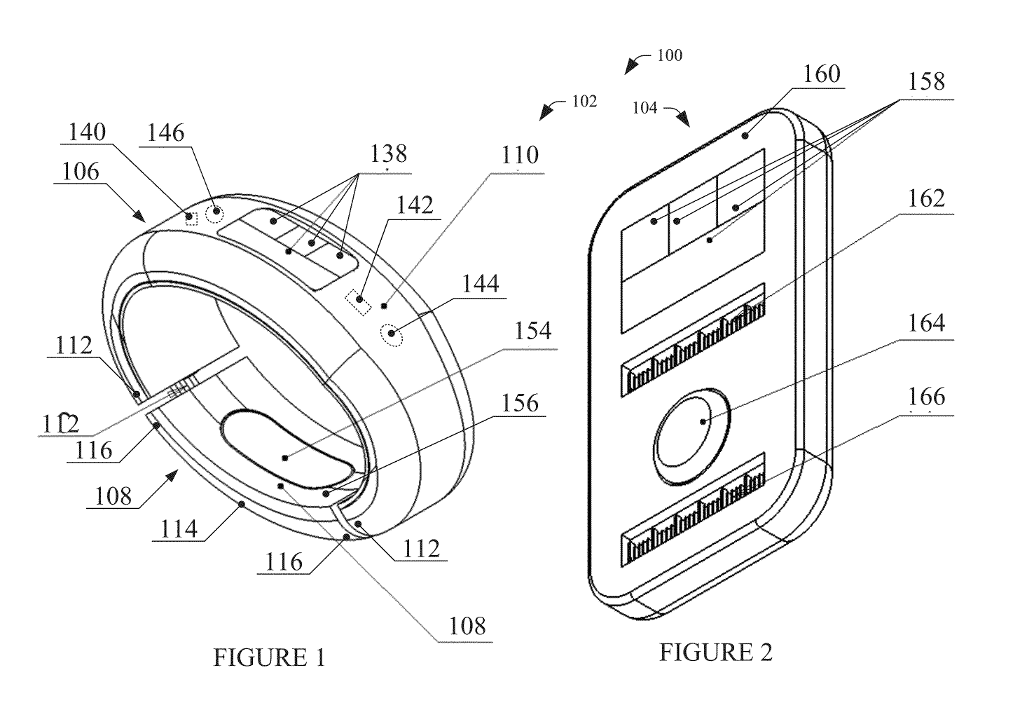

FIGS. 1 and 2 collectively schematically illustrate an apparatus 100 that includes a wearable device 102 and a remote controller 104. FIG. 1 schematically illustrates the wearable device 102. FIG. 2 schematically illustrates the remote controller 104. The wearable device 102, in the illustrated embodiment, is configured as a strap 102, which can be worn by a user. The remote controller 104 can be held by a user such as a guardian, a teacher and/or user.

With reference to FIGS. 1 and 2, the strap 102 includes an upper portion 106 and a lower portion 108. The upper portion 106 has a housing 110, which is curved (e.g., an open ellipse, circle, etc.) shaped, with end regions 112. The lower portion 108 has a housing 114, which is also curved shaped, with end regions 116.

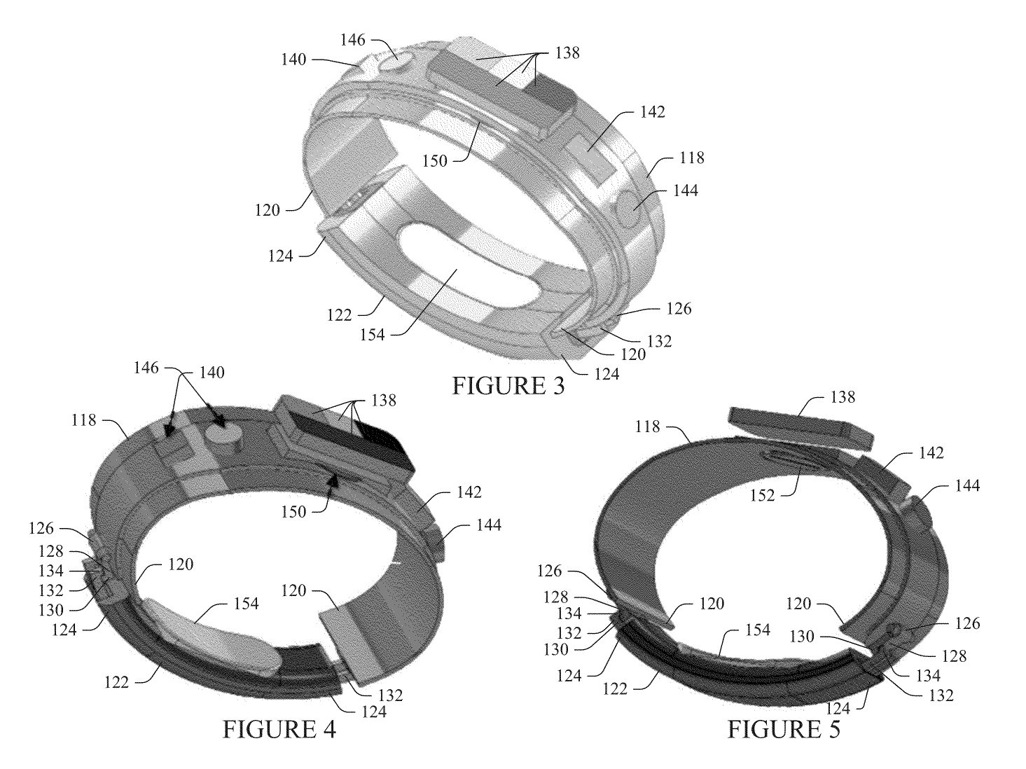

In FIGS. 3-5, the strap 102 is shown with the housings 110 and 114 transparent. The upper portion 106 includes an upper member 118, which is curved (e.g., an open ellipse, circle, etc.) shaped, with end regions 120. Each end region 120 includes a securing member 126 affixed thereto. Each securing member 126 includes a first element 128 affixed to the member 118 and a second element 130 protruding out from the first element 128 and an outer surface of the upper member 118 in a direction away from upper member 118. The lower portion 108 includes a lower member 122, which is curved (e.g., an open ellipse, circle, etc.) shaped, with end regions 124. The lower member 122 includes an elastic device 132 with end regions 134. Each end region 134 includes protruding portion 136 that protrudes out from the lower member 122.

The second elements 130 of the securing members 126 and protruding portions 136 of the elastic device 132 are configured such that when they engage each other, they mechanically connect together and form a closed (e.g., elliptical, circular, etc.) loop or ring. This can be achieved by stretching the elastic device 132, engaging the securing members 126 and protruding portions 136, and releasing the elastic device 132. An engaged elastic device 132 and securing members 126 can be disengage by again stretching the elastic device 132, disengaging the securing members 126 and protruding portions 136, and releasing the elastic device 132.

With reference to FIGS. 1-5, the upper portion 106 includes one or more buttons 138. The illustrated embodiment includes four (4) buttons, but other embodiments may include more or less buttons. The one or more buttons 138 are configured such that when pressed they actuate or to trigger corresponding buttons and/or lights (discussed below) on the remote controller 104 and/or a mobile application.

The upper portion 106 further includes integrated wireless technology 140 such as a built-in Bluetooth and a radio-frequency antenna. The integrated wireless technology 140 can be used to send information to the remote controller 104 and receive an instruction sent by the remote controller 104 sequentially or concurrently.

The upper portion 106 further includes a built-in vibration motor 142. The upper portion 106 further includes a controller 144 that controls the vibration motor 142. The controller 106 controls the vibration motor 142, for example, to generate vibrations in response to a trigger or activation signal from the remote controller 104 and/or automatically based on executable instructions store in memory of the controller 144.

The upper portion 106 further includes a built-in or integrated accelerometer 146, which may monitor the sudden and repeated actions (e.g., such as sudden onset of anxious behavior and repeated stereotyped behaviors) of the arms of the user, and detect the occurrence of outbursts or stereotypy of the user by means of the acceleration of the heart rate and action (preset values of the heart rate and acceleration, which may be obtained through multiple experiments), and reduce false alarms as much as possible. Users suffering from an outburst usually have high activity from the accelerometer.

In the illustrated embodiment, a power source 150 such as a button battery, a capacitor, etc. supplies power for the buttons 138, the integrated wireless technology 140, the vibration motor 142, the controller 106, and/or the accelerometer 146. The upper portion 106 includes a power source receiving region between the upper member 118 and the buttons 138. In other embodiments, the power source receiving region can be located elsewhere. Access to the power source receiving region, in the illustrated embodiment, is through a removable element 152, which may screw, snap, and/or otherwise engage and disengage, located on an inside of the upper member 118.

The lower portion 108 includes a built-in or integrated heart rate monitor 154. The heart rate monitor 154 is disposed at least partly on an inner side surface 156 of the lower portion 108, and contacts the skin of the user when worn. The heart rate monitor 154 can be used to analyze the heart rate of the user in real time and send the heart rate information to the remote controller 104, using the integrated wireless technology 140, which can display this information.

The remote controller 104 includes one or more buttons 158 on a face or panel 160 of the remote controller 104. In the illustrated embodiment, the buttons 158 are in the same layout (and optionally color) as the buttons 138 on the band 102. In other embodiments, the buttons may in include more or less buttons and/or have a different layout with respect to the buttons 138. The panel 160 further includes an accelerometer adjustment slider 162, which may be used to adjust a sensitivity of the built-in accelerometer 146 of the strap 102, in a remote control manner via the wireless technology 140.

The panel 160 further includes a control (e.g., a button, a touch sensitive surface, etc.) 164 configured to actuate the vibration controller 142 and hence invoke a vibration by the vibration motor 144 of the strap 102. In a variation, the control 164 includes a light source and lights up if physiological abnormalities are detected. The panel 160 further includes a slider 166 configured to control a strength of the vibration of the built-in vibration motor of the strap 102, in a remote control manner via the wireless technology 140.

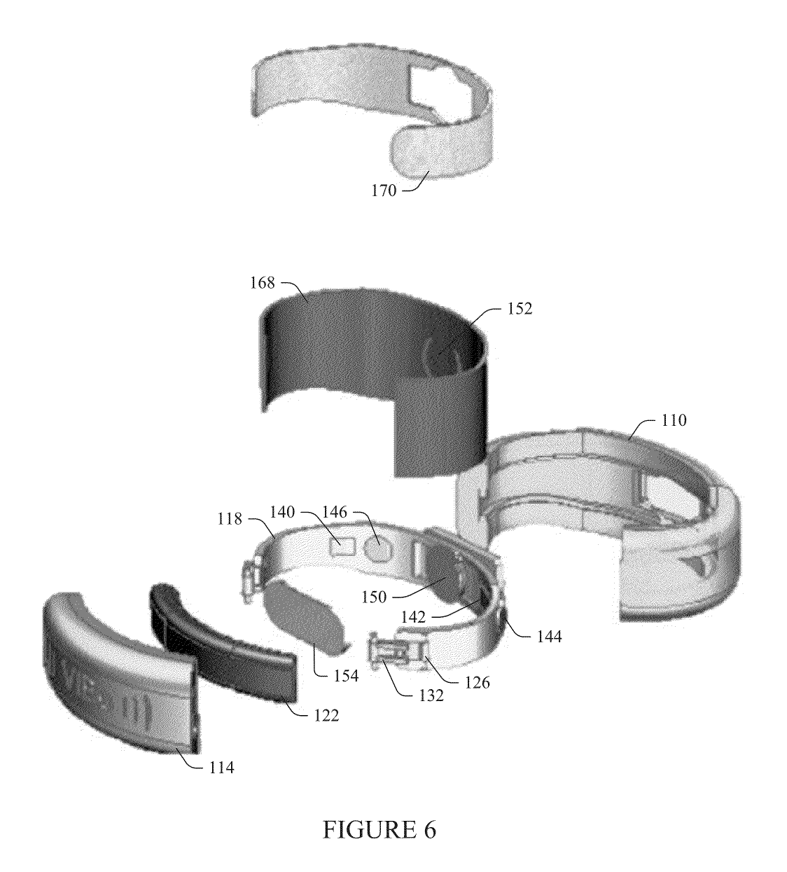

FIG. 6 shows an exploded view of some of the components of the wearable device 102. In this example, the wearable device 102 further includes components 168 and 170. The component 170 installs on an inner surface of the upper member 118. The component 170 includes a cutout in which the power source 150 is disposed. The component 168 installs on an inner surface of the component 170 and covers the power source 150. The removable element 152 removably attaches to the component 168.

A non-limiting example of a working model of the personal wearable device 100 for the autistic person comprises of: the strap, worn on the, and/or ankle, and/or neck of a user; the heart rate monitor, used to analyze the heart rate of the user and send notifications of irregularities in heart rate to the remote control using wireless technology; and the accelerometer, used to monitor the accelerations of the arms, legs or neck of the user and determine whether the user experiences an outburst according to the irregularities in acceleration or heart rate, measured by the accelerometer and the heart rate monitor respectively. Optionally, if physiological irregularities are detected, an alarm signal is sent to the remote controller, and meanwhile, the guardian controls the vibration motor and can adjust the vibration amplitude, sensitivity and pattern. If irregularities in physiological signals are not detected, the guardian may disregard the signal and resolve to not apply vibration.

Application example 1: when a user experiences an outburst and their heart rate increases, the heart rate monitor detects tachycardia and sends an alert to the remote control through a built-in radio-frequency antenna or Bluetooth in the strap, shown by the button (FIG. 2. 13) lighting up The heart rate indicator of the remote control can also inform the guardian of any abnormal activity of the user: the guardian can start the built-in motor in the strap through the vibration adjusting knob of the remote controller in order to generate vibrations on the wrist of the user to distract the attention of the user until the heartbeat of the user slows and the user is calm, at which time the guardian stops the vibration motor.

Application example 2: when in need of help, the user presses a certain pre-appointed button on the strap to express a need, the button of a corresponding color and position on the remote controller is lightened to inform the guardian of the child's needs, and the guardian takes actions meeting the need of the user according to which button was pressed.

What is claimed is:

1. A device for an autistic person, comprising:
a strap with a built-in heart rate monitor for analyzing irregularities in a heart rate of a subject in real time and sending alerts to a remote control through a signal transceiver if irregularities are detected, with the irregularities displayed by an indicator on the remote control;
wherein the strap is further provided with a built-in accelerometer for monitoring sudden and repeated actions of arms of the subject in order to determine whether the subject is undergoing an outburst according to a magnitude of an acceleration; and
the strap also includes a built-in vibration motor, which generates vibrations, with a remotely adjustable strength and sensitivity.
the strap can also include other sensors, such as, EDA (ElectroDermal Activity) that measures the emotional state of an individual.

2. The personal and wearable device for the autistic child of claim 1, wherein the strap further comprises an upper wrist strap and a lower strap, which are connected by an elastic band or Velcro.

3. The personal and wearable device for the autistic person of claim 2, wherein the upper strap comprises an upper surface with buttons with indicator lights, and the buttons are configured to trigger corresponding LEDs on a remote controller.

4. The personal and wearable device for the autistic child in claim 1, further comprising a remote controller with a panel with a sensitivity adjusting knob, which adjusts a sensitivity of the built-in accelerometer through a communications technology.

5. The personal and wearable device for the autistic child in claim 1, further comprising a remote controller with a panel with a vibration strength adjusting knob, which adjusts a strength of vibrations applied to the subject.

6. A method, comprising:
the strap is worn at a wrist, and/or an ankle, and/or a neck of the user;
monitoring a heart rate of a subject with a heart rate monitor of a strap to analyze irregularities in the heart rate of a user and send alerts to a remote control through a signal transceiver, wherein the alert is displayed on an indicator on the remote;
monitoring, with an accelerometer of the strap, actions of extremities of the user and determining whether the subject displays signs of an outburst based on accelerations and heart rate exceeding a predetermined threshold; and
in response to the accelerations or the heart rate exceeding the predetermined threshold, generating and transmitting an alarm signal to the remote controller, wherein, based on the alarm signal and a user input, the remote controller remotely adjusts a vibration amplitude of a vibration motor of the strap according to a strength of the alarm signal.

7. The working method of the device in claim 6, further comprising:
controlling the vibration to not vibrate in response to determining an outburst is not occurring even though the alarm signal is generate and transmitted.

8. The working method of the device in claim 6, further comprising:
receiving an input at the strap, during use, indicating a button press on the strap, which causes a light of a corresponding button of the remote controller to turn on.

9. The working method of the device in claim 6, further comprising:
receiving an input at the strap indicating a button press and invoking an LED, or other such indicator, of a corresponding to the button on the remote controller to light up.

10. An apparatus, comprising:
a device, including:
an upper portion with a set of controls, an interface, an accelerometer, a vibration motor, and a vibration motor controller; and
a lower portion with a heart rate monitor;
wherein the upper portion and lower portions are configured to engage at end regions to provide a closed ring through complementary securing members and disengage through the complementary securing members.

11. The apparatus of claim 10, wherein the set of controls includes a light source.

12. The apparatus of claim 10, further comprising:
a remote control, including:
a corresponding set of controls;
a complementary interface, wherein the interface and the complementary interface are configured to communicate with each other;
an accelerometer control configured to control the sensitivity of the accelerometer of the device; and
a vibration motor control configured to invoke the vibration motor controller to vibrate the vibration motor.

13. The apparatus of claim 12, wherein the vibration motor control configured to control the vibration strength of the vibration motor.

14. The apparatus of claim 12, wherein the heart rate monitor monitors a heart rate of a user and transmits a heart signal to the complementary interface of the remote control.

15. The apparatus of claim 14, wherein the heart rate transmits, via the interface, an alert signal in response to the heart rate exceeding the predetermined heart rate threshold. This predetermined threshold is continuously monitored and automatically adjusted based on the user activity.

16. The apparatus of claim 14, wherein the remote control displays the heart rate of the user based on the heart rate signal.

17. The apparatus of claim 12, wherein the corresponding set of controls includes a light source and/or other notification medium.

18. The apparatus of claim 17, wherein a control of the set of controls is configured to light up in response to being actuated, and the actuating the control invokes a corresponding control of the set of corresponding controls to light up.

19. The apparatus of claim 12, wherein the accelerometer transmits, via the interface, an alert signal in response to a sensed motion exceeding a predetermined motion threshold.

20. The apparatus of claim 12, wherein the vibration motor control transmits, via the complementary interface, a vibration control signal to the interface of the device in response to at least one of a motion sensed by the accelerometer or a heart rate sense by the heart rate monitor exceeding a predetermined motion threshold, and the vibration motor controller, in response to receiving the vibration control signal, controls the vibration motor to vibrate at a predetermine level.
